(19) United States
(12) Plant Patent Application Publication
(10) Pub. No.: US 2017/0303457 P1
(43) Pub. Date: Oct. 19, 2017

(54) Ornithogalum plant named 'BCOR -09.001'

(50) ;

(71) Applicant: Rodolfo Valdoz Bautista, Half Moon Bay, CA (US)

(72) Inventor: Rodolfo Valdoz Bautista, Half Moon Bay, CA (US)

(21) Appl. No.: 14/999,281

(22) Filed: Apr. 18, 2016

(51) Int. Cl. A01H 5/02

(52) U.S. Cl. CPC A01H 5/0222
USPC PLT/462

(57) ABSTRACT

A new and distinct cultivar of Ornithogalum dubium named ‘BCOR-09.001’ resulting from a controlled hybridization. The new cultivar ‘BCOR-09.001’ has R.H.S. 24 A (orange group) tepals at maturity, erect and strong racemes which resist bending under low light conditions, a long blooming habit and broad, channeled leaves.

Botanical classification: Ornithogalum dubium ‘BCOR-09.001’.

Variety denomination: ‘BCOR-09.001’.

### BACKGROUND OF THE INVENTION

This invention relates to a new and distinct cultivar of the Lilaceae family. The botanical name of the plant is Ornithogalum dubium ‘BCOR-09.001’.

The new cultivar is the result of controlled crossing of commercial varieties.

The new variety ‘BCOR-09.001’ is differentiated from other commercial varieties known to the inventor as shown in the table below.

TABLE 1

| | ‘Lourdes’ | | ‘Chesapeake | | |
|---|---|---|---|---|---|
| | Unpatented | ‘Bethlehem’ | Snowflake’ | ‘Orangezicht’ | |
| | Commerical | U.S. Plant Pat. | U.S. Plant Pat. | U.S. Plant Pat. | New Variety |
| | Variety | No. 21,427 | No. 13,200 | No. 12,226 | BCOR-09.001 |
| Rachis/Raceme | 40 to 50 cm | 43 cm | 20-30 cm | 26 cm | 40 to 50 cm |
| height | | | | | |
| Rachis color | Varies between | R.H.S. 146A | Not reported | Not reported | Mature stem |
| | R.H.S. 146B, | | | | R.H.S. 146A |
| | R.H.S. 147B, | | | | (green group) |
| | or | | | | Immature stem |
| | R.H.S. 145D | | | | R.H.S. 146D |
| | | | | | (yellow-green |
| | | | | | group) |
| Reflexed | No | Yes | Not reported | Leaves lay flat | Most upright; |
| leaves | | | | on ground | some slightly |
| | | | | | reflexed |
| Leaf color | R.H.S. 137A | R.H.S. 146A | R.H.S. 143B | Upper surface | Immature |
| | | | | R.H.S. 137A | 137C (green |
| | | | | or 147A | group); Mature |
| | | | | | 144A (yellow- |
| | | | | | green group) |
| Floret diameter | 30 mm | 70 mm | 50-60 mm | 50-60 mm | 40 mm |
| Tepal height | 30 mm | 36 mm | 20-25 mm | 25-27 mm | 25-27 mm |
| Tepal Color | R.H.S. 25A | R.H.S. 155C | R.H.S. 155A-D | Varies | R.H.S. 24A |
| | | | | between | |
| | | | | R.H.S. 25A, | |
| | | | | R.H.S. 25D, or | |
| | | | | R.H.S. 28A | |
| Tepal Spot | R.H.S. 152B | R.H.S. 152A | R.H.S. 147A | R.H.S. 200B | R.H.S. 152A |
| Color | | | | | |

The new variety is suitable for commercial sale as a potted plant.

The new cultivar ‘BCOR-09.001’ has been asexually reproduced under controlled environmental conditions at a nursery in Half Moon Bay, Calif. under the direction of the inventor with its distinguishing characteristics remaining stable.

Asexual reproduction was first accomplished when vegetative cuttings were taken from the initially selected plant. Examination of asexually reproduced, successive generations grown in a nursery at Half Moon Bay, Calif. show that the combination of characteristics as herein disclosed for ‘BCOR-09.001’ remains firmly fixed through three generations.

### DESCRIPTION OF THE DRAWINGS

The accompanying drawings consist of color photographs that show the typical plant form, including the inflorescence, foliage, and sepals.

FIG. 1 is a view of the new variety showing its form.

FIG. 2 is a top view of two inflorescences of the new variety.

FIG. 3 is a close-up view of the base of the plant of the new variety.

FIG. 4 is a close-up view of the top of the inflorescence of the new variety.

FIG. 5 is a close-up view of a floret of the new variety.

FIG. 6 is a close-up view of an inflorescence of the new variety.

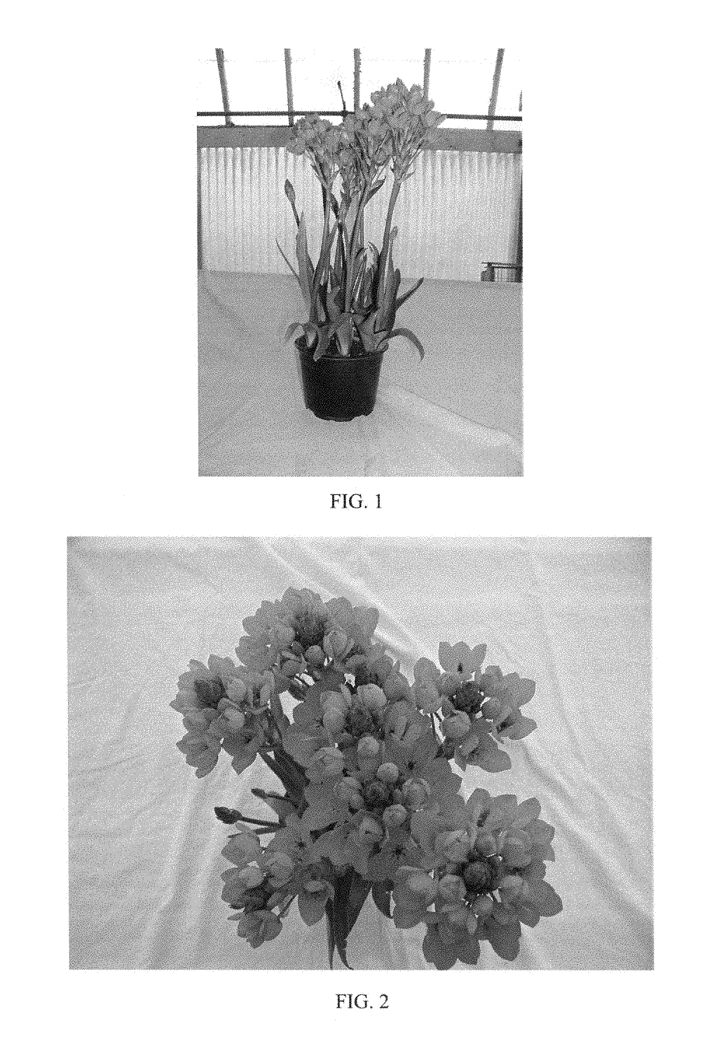

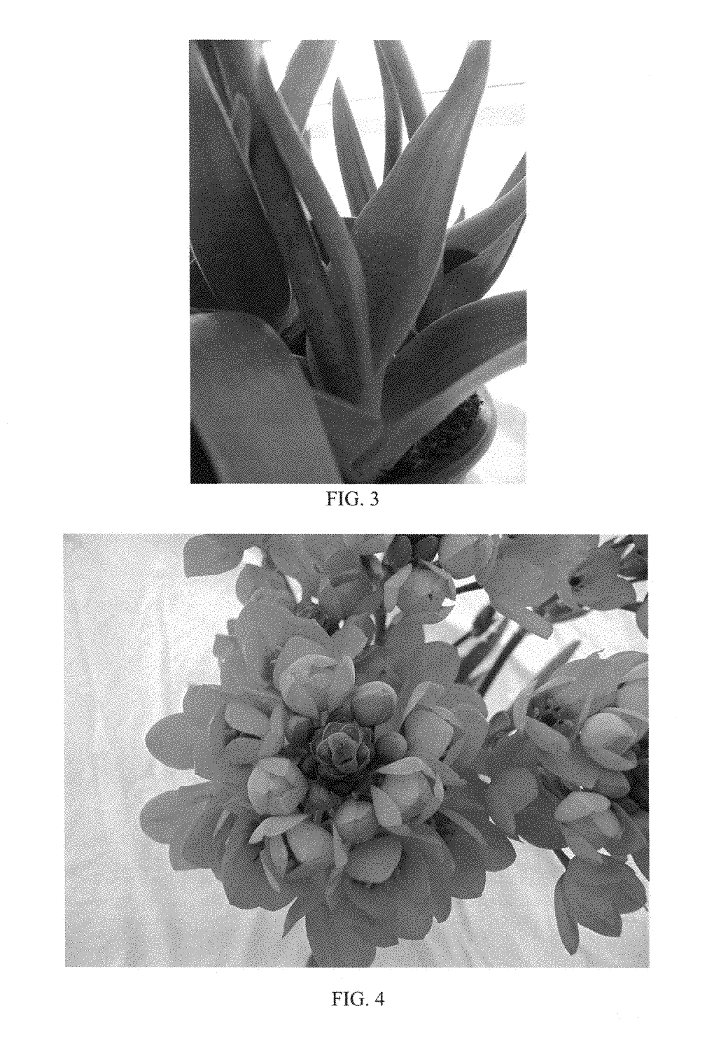

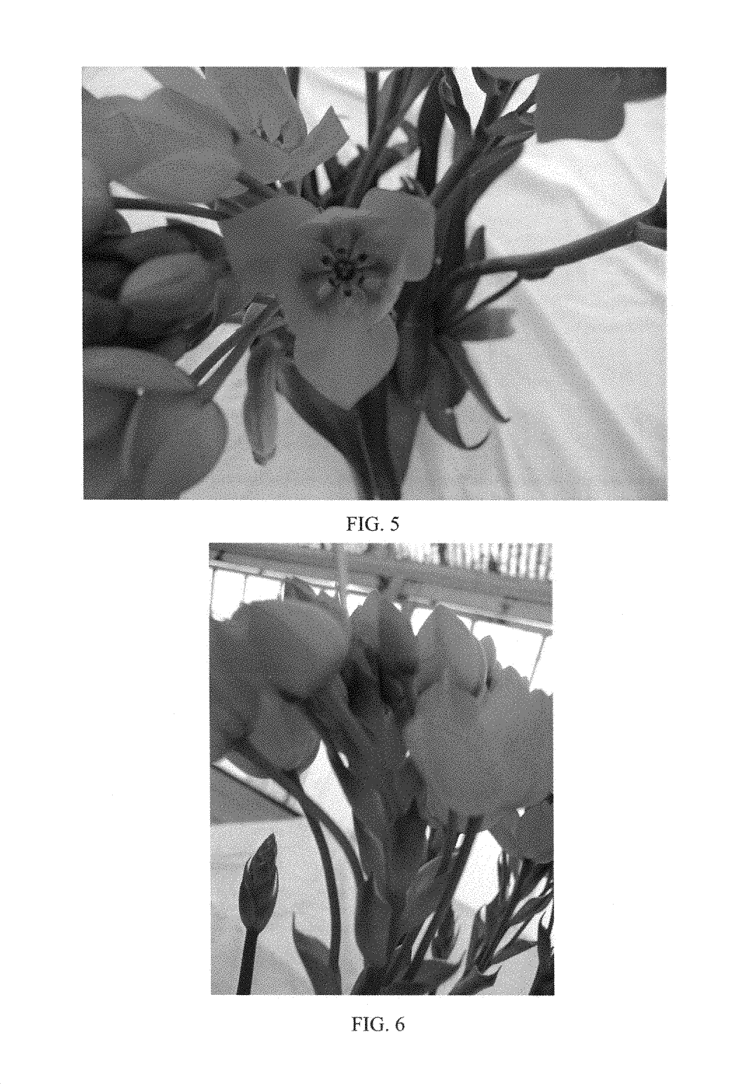

### DESCRIPTION OF THE NEW PLANT

The plants shown in the figures were grown in 6″ pots. The plants were started in a greenhouse from leaf cuttings.

‘BCOR-09.001’ has not been observed under all possible environmental conditions. The phenotype may vary significantly with variations in environment such as temperature, light intensity and day length. Color determinations were made with The Royal Horticultural Society (R.H.S.) Colour Chart.
The plant:
   Origin.—Controlled cross of commercial varieties.
   Form.—Spreading basal leaves with upright inflorescences on tall rachis. A typical plant with a mature inflorescence that is ready for sale at ten months is approximately 40 to 50 cm high when grown in a 6″ pot with appropriate soil amendments. Plants shown are 47 cm high.
   Growth.—Upright rachis with upright, leaves. Inflorescence is large and showy.
   Rachis.—The rachis is upright and glaucous. Its color varies between R.H.S. 146 D (yellow-green group) when younger and R.H.S. 146 A (yellow-green group), at maturity of the inflorescence. Diameter is 9 mm. The length of the rachis to the first floret and bract pair is 30 cm. Rachis or stem at its base is 10 mm in diameter.
   Foliage.—Usually three to five leaves surround each rachis on a mature plant ready for commercial sale. Leaves are whorled at base of stem. Shape of leaf. — Fleshy, channel-shaped, lanceolate. Margins are entire. Apex is acute. Texture. — Glaucescent. Color. — Mature leaves are R.H.S. 144 A (yellow group) on both abaxial and adaxial surfaces. Large leaves are 35 cm long and 4.5 cm wide. Immature leaves are R.H.S. 137 C (green group).
Inflorescence:
   Form.—Corymbose racemes. Indeterminate. Approximately 50 or more individual florets per inflorescence on pedicels that elongate as the florets opens, and on average are as long as 120 mm for mature florets. Apical buds on developed inflorescences are predominately green — R.H.S. 145 D (yellow-green group), with their apex being R.H.S. 146 B (yellow-green group), 1.2-1.5 cm tall and have diameter of 5 mm wide. The rachis for the inflorescence is strong and upright. The pedicels are 3 mm wide, glabrous and similar in color to the rachis at their point of attachment.
   Size of Inflorescence.—Large. A typical inflorescence at this age, as shown in the figures can be 15 cm high.
   Shape.—Basal florets opening first with pedicels that elongate that give the raceme a corymbose appearance as the inflorescence ages.
   Appearance.—Showy.
Florets:
   General.—The florets at the base of the inflorescence open first, and open more and more as the inflorescence ages. Florets are perfect and complete. Perianth is not keeled. Each floret is associated with a bract that can be as wide as 20 mm and as high as 27 mm when floret is beginning to open, and has tips that are R.H.S. 137 B (green group). Tips of bract are darkest part of bract. Color of bracts fade as inflorescence ages and bracts continue to elongate. The bracts are ovate with narrowly acute apexes and relatively wide bases and reflexed/curled around radius. Perianth: There are 6 ovate tepals that are predominately R.H.S. 24 A (orange group) on their abaxial and adaxial surfaces. Tepals have a spot at their base. The color of the spot on the adaxial surface is R.H.S. 152A (yellow-green group). Color of the spot on the abaxial surface spot is R.H.S. 143 A (green group). Spot fades as floret ages. Floret diameter: 40 mm. Tepals of mature florets are typically 25-27 mm high, 20 mm wide at their widest point, and 5 mm wide at their attachment. The spot is most prominent on the immature tepals where the spotted base is approximately 5 mm high and extends the width of the tepal at the base. Apex shape of the tepal is abruptly acuminate.
   Stamens.—6 stamens (two sets of 3). Pollen is R.H.S. 14 A (yellow-orange group). Plant produces abundant pollen. Filament is wide with basal wiring. Filament is 3 mm wide at base and 4 mm high. Anther is 3 mm high and basally attached. Filament base is R.H.S. 155 A (white group). Anther is R.H.S. 152 B (yellow-green group) before opening to reveal pollen.
   Stigma.—Generally, a three-pronged stigma on one pistil per floret. The style is 2 mm long. The stigma is approximately 2 mm long.
   Ovary.—The ovary is three-celled with axile placentae. The top of the ovary is R.H.S. 139 A (green group). The ovary is 8 mm high and has a diameter of 4 mm.
   Fragrance.—Florets product a very mild citrus-like fragrance, if any.

I claim:

1. A new and distinct Ornithogalum plant named ‘BCOR-09.001’ substantially as herein shown and described.
